(19) United States
(12) Patent Application Publication
(10) Pub. No.: US 2012/0165615 A1
(43) Pub. Date: Jun. 28, 2012

(54) APPARATUS AND METHOD FOR TELEMEDICINE

(76) Inventor: Kyuhyoung Choi, Seoul (KR)

(21) Appl. No.: 12/976,804

(22) Filed: Dec. 22, 2010

(51) Int. Cl. A61B 5/00

(52) U.S. Cl. 600/300

(57) ABSTRACT

The present invention provides an apparatus and a method for telemedicine and, more particularly, to an apparatus and a method for telemedicine capable of providing a patient image and various types of information helping medical treatment.

### BACKGROUND OF THE INVENTION

1. Field of the Invention

The present invention relates to an apparatus and a method for telemedicine. More particularly, the present invention relates to an apparatus and a method for telemedicine capable of providing a patient image and various types of information helping medical treatment.

2. Description of the Related Art

Recently, the social interest in telemedicine is rapidly growing. In particular, as remote video medical treatment is available, telemedicine is anticipated to gradually replace clinic-based treatment. However, telemedicine is in the stage of a simple video medical treatment, it is not sufficient to attain effective medical treatment.

### BRIEF SUMMARY OF THE INVENTION

The problems to be solved by the present invention are as follows.

An aspect of the present invention provides an apparatus and a method for telemedicine capable of providing various types of information helping a remote medical treatment.

Another aspect of the present invention provides an apparatus and a method for telemedicine capable of providing information helping treatment such as biometric information, or the like, in the event of telemedicine through an intuitive interface.

Another aspect of the present invention provides an apparatus and a method for telemedicine capable of providing medical treatment information of the past in the event of telemedicine.

Another aspect of the present invention provides an apparatus and a method for telemedicine capable of providing medical treatment records between doctors to allow the doctors to share them.

The problems are solved by providing an apparatus and a method for telemedicine.

According to an aspect of the present invention, an apparatus for telemedicine comprises: an output module configured to output a patient image; an input module configured to receive a signal for selecting a body part; and a controller configured to obtain biometric information related to the body part, and control the output module to output the biometric information at a position corresponding to the body part on the patient image.

According to another aspect of the present invention, an apparatus for telemedicine comprises: an output module configured to output a patient image; an input module configured to receive a signal selecting a position corresponding to a body part on the patient image; and a controller configured to obtain biometric information related to the body part, and control the output module to output the biometric information at the body part on the patient image.

According to another aspect of the present invention, a method for telemedicine comprises: outputting a patient image; receiving a signal selecting a body part from the outputted patient image; obtaining biometric information related to the body part; and outputting the biometric information to a position corresponding to the body part on the patient image.

According to another aspect of the present invention, an apparatus for telemedicine comprises: an input module; an output module; a storage unit configured to store at least one prior medical treatment information; and a controller configured to control the output module to display at least one date corresponding to the at least one prior medical treatment information, receive, via the input module, a signal for selecting a date, and control the output module to display prior medical treatment information corresponding to the selected date.

According to another aspect of the present invention, an apparatus for telemedicine comprises: an input module; an output module; a storage unit configured to store at least one prior medical treatment information; and a controller configured to control the output module to display at least one date corresponding to the at least one prior medical treatment information, receive, via the input module, a signal for selecting a date, and control the output module to display prior medical treatment information corresponding to the date. Here, the prior medical treatment information includes biometric information measured from a patient at the corresponding date, and the controller controls the output module to output a patient image and output biometric information to a position corresponding to a body part related to biometric information on the patient image.

According to another aspect of the present invention, an apparatus for telemedicine comprises: an output module; a communication module configured to communicate with an external device; a storage unit configured to store prior patient information; and a controller configured to receive, via the communication module, patient information from a patient terminal, determine whether or not the patient is new based on the prior patient information and the patient information, when the patient is new, transmit, via the communication module, a signal for requesting prior medical treatment records to the patient terminal, receive, via the communication module, the prior medical treatment records from the patient terminal, and control the output module to output the received prior medical treatment records.

According to another aspect of the present invention, an apparatus for telemedicine comprises: an output module; a communication module configured to communicate with an external device; a storage unit configured to store prior patient information; and a controller configured to receive, via the communication module, patient information from a patient terminal, determine whether or not the patient is new based on the prior patient information and the patient information, when the patient is new, transmit, via the communication module, a signal for requesting prior medical treatment records to the patient terminal, receive, via the communication module, the prior medical treatment records from the patient terminal, and control the output module to output the received prior medical treatment records. Here, the patient terminal transmits a signal for requesting prior medical treatment records and authentication information to a different apparatus for telemedicine, and when the authentication information is correct, the different apparatus for telemedicine transmits the prior medical treatment records to the patient terminal.

The present invention has the following advantages.

According to an exemplary embodiment of the present invention, because biometric information of a patient is provided along with an image of the patient, the doctor can more effectively perform telemedicine.

According to an exemplary embodiment of the present invention, because the patient's prior medical treatment history and prior medical treatment information are provided, the doctor can review content of the prior medical treatment and more precisely treat the patient.

According to an exemplary embodiment of the present invention, because the doctor can receive medical treatment records of a different doctor when he first treats the patient, the doctor can easily recognize the condition of the patient's health (or the patient's physical condition).

According to an exemplary embodiment of the present invention, in the event of telemedicine, comprehensive information can be provided to the doctor via an intuitive interface, so the doctor's convenience in telemedicine can be improved.

According to an exemplary embodiment of the present invention, telemedicine can become active, so a medical service as good as a clinic-based treatment can be provided from a remote area.

According to an exemplary embodiment of the present invention, individuals can live a healthier life and social costs for unnecessarily wasted medical treatment can be reduced.

The foregoing and other objects, features, aspects and advantages of the present invention will become more apparent from the following detailed description of the present invention when taken in conjunction with the accompanying drawings.

### BRIEF DESCRIPTION OF THE DRAWINGS

The present invention will become more fully understood from the detailed description given hereinbelow and the accompanying drawings which are given by way of illustration only, and thus are not limitative of the present invention and wherein:

FIG. 1 is a view illustrating a system for telemedicine according to an exemplary embodiment of the present invention;

FIG. 2 is a perspective view of an apparatus for telemedicine according to an exemplary embodiment of the present invention;

FIG. 3 is a schematic block diagram of the apparatus for telemedicine according to an exemplary embodiment of the present invention;

FIG. 4 is a view illustrating an input module and an output module of the apparatus for telemedicine according to an exemplary embodiment of the present invention;

FIG. 5 is a view illustrating a communication module of the apparatus for telemedicine according to an exemplary embodiment of the present invention;

FIG. 6 is a flow chart illustrating the process of a method for telemedicine according to a first exemplary embodiment of the present invention;

FIG. 7 is a first view of outputting an image reflecting a patient in the method for telemedicine according to the first exemplary embodiment of the present invention;

FIG. 8 is a second view of outputting an image reflecting a patient in the method for telemedicine according to the first exemplary embodiment of the present invention;

FIG. 9 is a view showing an input of a signal for selecting a body part in the method for telemedicine according to the first exemplary embodiment of the present invention;

FIG. 10 is a first view showing an input of biometric information in the method for telemedicine according to the first exemplary embodiment of the present invention;

FIG. 11 is a second view showing an input of biometric information in the method for telemedicine according to the first exemplary embodiment of the present invention;

FIG. 12 is a flow chart illustrating the process of a method for telemedicine according to a second exemplary embodiment of the present invention;

FIG. 13 is a view showing an output of prior medical treatment history in the method for telemedicine according to the second exemplary embodiment of the present invention;

FIG. 14 is a view showing an output of prior medical treatment records in the method for telemedicine according to the second exemplary embodiment of the present invention;

FIG. 15 is a flow chart illustrating the process of a method for telemedicine according to a third exemplary embodiment of the present invention; and

FIG. 16 is a signal flow chart of a system for telemedicine in the method for telemedicine according to the third exemplary embodiment of the present invention.

### DETAILED DESCRIPTION OF THE INVENTION

The terminology used herein is for the purpose of describing particular embodiments only and is not intended to be limiting of the invention.

The present invention may be embodied in many different forms and may have various embodiments, of which particular ones will be illustrated in drawings and will be described in detail. However, it should be understood that the following exemplifying description of the invention is not meant to restrict the invention to specific forms of the present invention but rather the present invention is meant to cover all modifications, similarities and alternatives which are included in the spirit and scope of the present invention.

The same reference numerals will be used throughout to designate the same or like components, and a repeated description may be omitted.

The present invention is not limited to the exemplary embodiments described hereinafter.

Exemplary embodiments of the present invention will now be described with reference to the accompanying drawings.

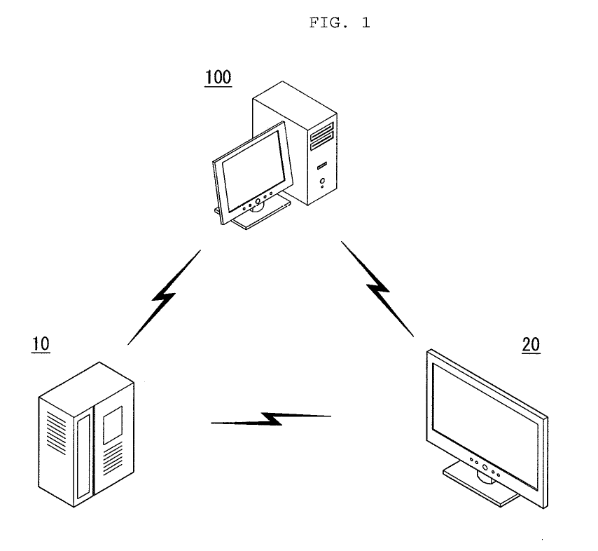

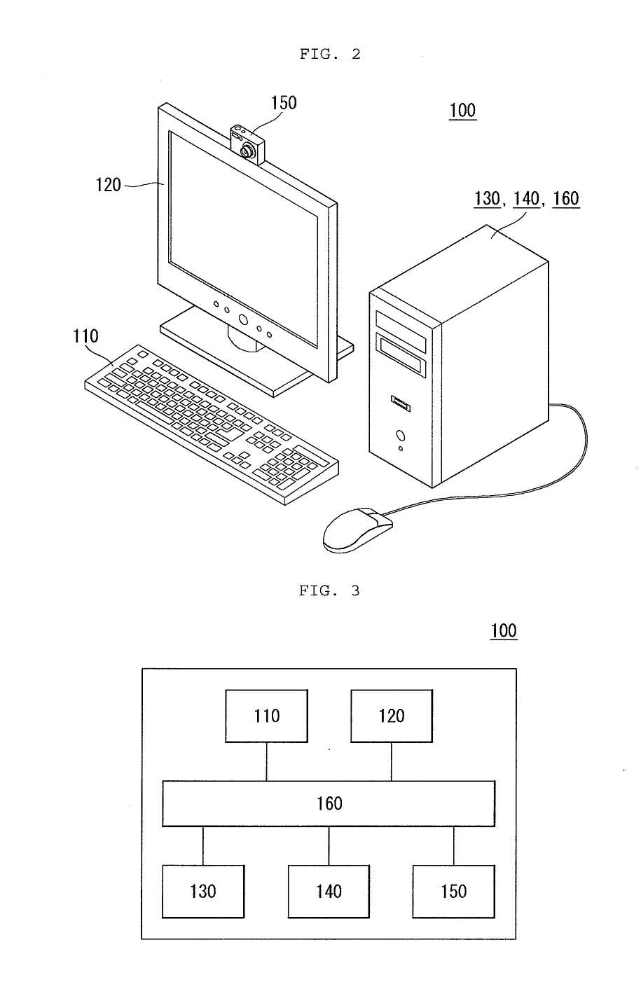

An apparatus for telemedicine 100 according to an exemplary embodiment of the present invention will now be described with reference to FIGS. 1 to 3. FIG. 1 is a view illustrating a system for telemedicine according to an exemplary embodiment of the present invention, FIG. 2 is a perspective view of an apparatus for telemedicine according to an exemplary embodiment of the present invention, and FIG. 3 is a schematic block diagram of the apparatus for telemedicine according to an exemplary embodiment of the present invention.

The apparatus for telemedicine 100 may enable the doctor to perform telemedicine. Here, the telemedicine refers to doctor's treating a patient who is at a remote area. Also, here, the doctor is a broad concept including all the medical parties substituting doctor, as well as a general practitioner. The medical parties may include, but are not limited thereto, a specialized doctor, a veterinarian, a pharmacist, a nurse, a paramedic, and a health manager. Also, the patient does not refer to only a person who becomes ill but includes a person who receives medical treatment. Also, the patient is not necessarily limited to a person. Also, medical treatment may include any consultation regarding the condition of the patient's health as well as medical treatment of a general concept. For example, a process of seeking an advice of a health trainer for body shaping may be included in medical treatment.

The telemedicine may be performed through a system for telemedicine comprising at least one of an apparatus 100 for telemedicine, a patient terminal 20, and a server 10. In the system for telemedicine, the apparatus 100 for telemedicine, the patient terminal 20, and the server 10 may exchange various types of information related to medical treatment through communication, and provide the same to the doctor or the patient to thus allow perform telemedicine.

For example, the apparatus 100 for telemedicine may receive an image of the patient from the patient terminal 20 and provides the same to the doctor in real time, and capture an image of the doctor and provide the same to the patient terminal 20. Conversely, the patient terminal 20 may capture an image of the patient and provide the same to the apparatus 100 for telemedicine, and receive an image of the doctor from the apparatus 100 for telemedicine and provide the same to the patient in real time. Also, in this case, the server 10 may provide various types of information regarding the patient's health to a doctor terminal or to the patient terminal 20 to ensure effective medical treatment.

As shown in FIGS. 2 and 3, the apparatus 100 for telemedicine may comprise at least one of an input module 110 receiving various signals from a user, e.g., the doctor, an output module 120 providing various types of information regarding medical treatment to the user, a communication module 130 performing communication with various external devices, such as the patient terminal 20, the server 10, or the like, related to telemedicine, a storage unit 140 storing information regarding medical treatment, an camera 150 capturing an image of the doctor, and a controller 160 controlling elements of the apparatus 100 for telemedicine to perform telemedicine. In the apparatus 100 for telemedicine according to an exemplary embodiment of the present invention, the foregoing elements are not essential, and the apparatus 100 for telemedicine may selectively comprise the foregoing elements.

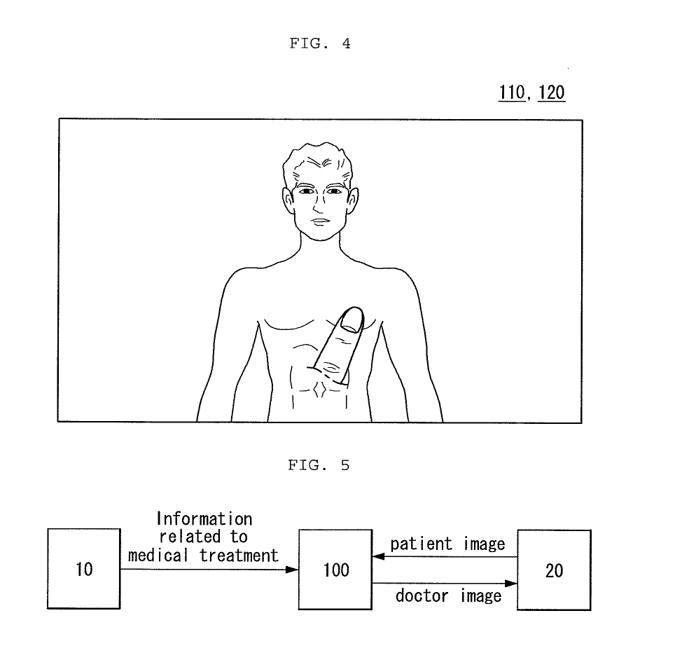

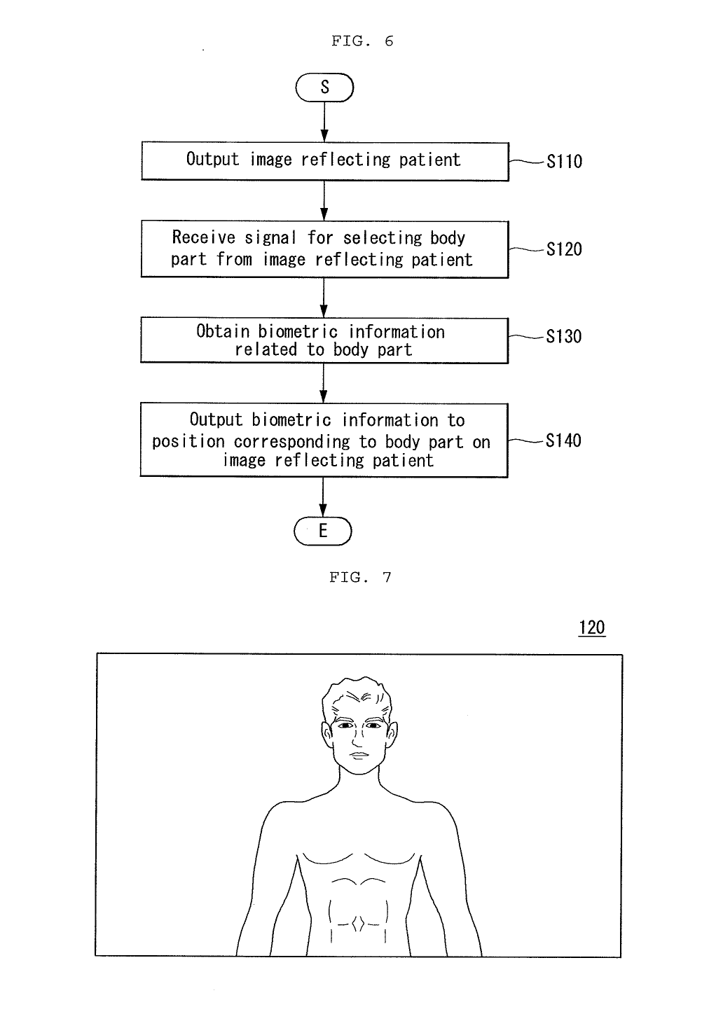

Hereinafter, the elements of the apparatus 100 for telemedicine will be described in order of the input module 110, the output module, the communication module 130, the storage unit 140, the camera 150 and the controller 160. FIG. 4 is a view illustrating an input module and an output module of the apparatus for telemedicine according to an exemplary embodiment of the present invention, and FIG. 5 is a view illustrating a communication module of the apparatus for telemedicine according to an exemplary embodiment of the present invention.

The input module 110 may receive a signal from an external source. The input module 110 may include a keyboard, a mouse, a touch screen, a microphone, and at least one of various other input devices obvious to a skilled person in the art.

For example, as shown in FIG. 4, the input module 110 may be provided as a touch screen. Here, the touch screen may display an image reflecting a patient and receive a touch signal regarding a position corresponding to a particular body part of the patient on the displayed image from the user.

The output module 120 may output information to the exterior. Thus, the user can receive the information from the apparatus 100 for telemedicine. The output module 120 may output information in various manners. The output module 120 may output the information by using a visual signal, an audible signal, and a tactile (or haptic) signal. The output module 120 may include a display, a speaker, a vibrator, and the like. The display may display information processed in the apparatus 100 for telemedicine. The display may be provided as a liquid crystal display, a thin film transistor-liquid crystal display, an organic light-emitting diode, a flexible display, a 3D display, and the like.

The output module 120 is not limited to the foregoing examples and may include various other devices obvious to the skilled person in the art.

The output module 120 may provide various types of information related to medical treatment to the user. For example, as shown in FIG. 4, the output module 120 may display an image of a patient in real time. Thus, the user, namely, the doctor, may perform telemedicine upon viewing the real time image of the patient.

The communication module 130 may perform communication with an external device. The communication module 130 may perform communication with the external device through wired/wireless network in various manners. For example, the communication module 130 may perform wireline communication such as a universal serial bus (USB), an RS-232 scheme, and the like. In another example, the communication module 140 may perform wireless communication such as Wi-Fi, WiBro, Bluetooth™, ZigBee™, RFID, IrDA, and the like. The method according to which the communication module 140 performs communication is not limited to the foregoing examples but may include various other methods known to the skilled person in the art.

The communication module 130 may perform communication with various external devices, and the external devices may include the patient terminal 20, the server 10, a different apparatus 30 for telemedicine (FIG. 16), a personal computer, a mobile communication terminal, a hospital device, a drug store device, an Internet site, and the like. For example, as illustrated in FIG. 5, the communication module 130 may receive an image of the patient from the patient terminal 20 in real time and allow the apparatus 100 for telemedicine to provide the image of the patient to the doctor. In another example, as shown in FIG. 5, the communication module 130 may receive information regarding the patient's body from the server 10 and provide the received information to the doctor. Upon receiving the information, the doctor may utilize the information for medical treatment.

The storage unit 140 may store information. The storage unit 140 may store the information temporarily or semi-permanently. The storage unit 140 may include various storage mediums. For example, the storage unit 140 may include a flash memory, a RAM (random access memory), an SRAM (static random access memory, a ROM (read only memory), an EEPROM (electrically erasable programmable read only memory), a hard disk, a magnetic memory, a magnetic disc, and an optical disk such as a CD or a Blu-ray disk, a card type memory such as an SD card, and any other storage medium obvious to the skilled person in the art. The storage unit 140 may be installed within the apparatus 100 for telemedicine, may be configured to be detachably mounted in the apparatus 100 telemedicine, or may be separately positioned outside the apparatus 100 telemedicine.

The storage unit 140 may store information regarding medical treatment. The storage unit 140 may obtain information from the input module 110, the communication module 130, the controller 160, and the like, and store the same. Here, the information regarding medical treatment may include medical treatment history (or background), medical treatment records, personal information regarding the patient, physical information of the patient, medication records of the patient, information regarding the doctor for medical treatment, diagnosis, prescription information, a medical treatment image, physical examination information, and the like. The physical information of the patient may include information regarding the user's gender, age, height, weight, medical history, blood sugar level, blood pressure level, and other information regarding the patient's body. The prescription information may include information regarding a prescription date, a prescription doctor (i.e., the doctor who has prescribed), a prescribed patient, a prescribed medicine, the directions of the prescribed medicine, and the like. The physical examination information may include biometric information measured by examining the patient.

The camera 150 may capture an image of an object to obtain the image. The camera 150 may be implemented as the camera 150, a camera, or the like, that captures an optical image, to thus obtain a still image or a video. For example, the camera 150 may capture an image of the doctor to obtain the image of the doctor. The apparatus 100 for telemedicine provides the image captured by the camera 150 to the patient terminal 20, and the patient terminal 20 may provide the image of the doctor to the patient in real time. Thus, telemedicine may be performed through the real time video.

The controller 160 may control the general operation of the apparatus 100 for telemedicine to perform telemedicine, and the other elements of the apparatus 100 for telemedicine. For example, the controller 160 may associate various types of information and perform controlling and processing to allow the information to be used. The controller 160 may be implemented as a computer or a similar device by using software, hardware, and their combination.

For hardware implementation, the controller 160 may include at least one of ASICs (application specific integrated circuits), DSPs (digital signal processors), DSPDs (digital signal processing devices), PLDs (programmable logic devices), FPGAs (field programmable gate arrays, processors, controllers, micro-controllers, microprocessors, and an electric device that performs a control function obvious to the skilled person in the art.

For software implementation, the controller 160 may be implemented by software written by a proper program language or a software application. The software may be stored in the storage unit 140 and executed by a hardware configuration. Also, the software may be transmitted from an external device, e.g., the server 10, or the like, to the apparatus 100 for telemedicine, so as to be installed in the apparatus 100 for telemedicine.

Details of the controller 160 will be described in explaining a method for telemedicine according to an exemplary embodiment of the present invention.

The method for telemedicine according to an exemplary embodiment will now be described.

The method for telemedicine according to an exemplary embodiment will now be described by using the apparatus 100 for telemedicine according to an exemplary embodiment. Here, the apparatus 100 for telemedicine according to an exemplary embodiment is used to easily explain the method for telemedicine according to an exemplary embodiment. Thus, the method for telemedicine according to an exemplary embodiment is not limited by the apparatus 100 for telemedicine according to an exemplary embodiment.

The method for telemedicine according to an exemplary embodiment may be performed by using a different device having the same function as that of the apparatus 100 for telemedicine according to an exemplary embodiment.

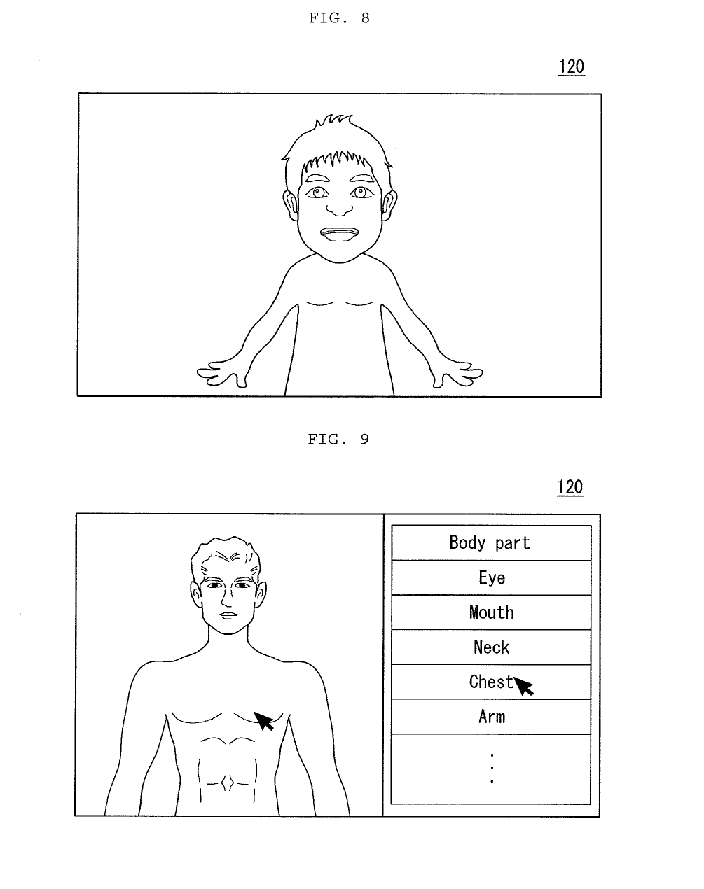

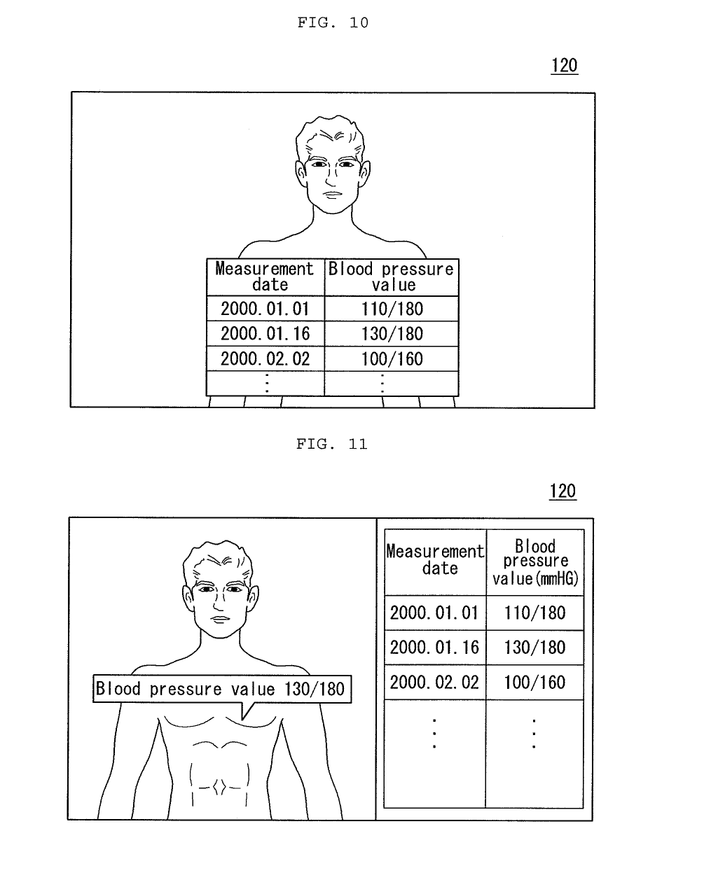

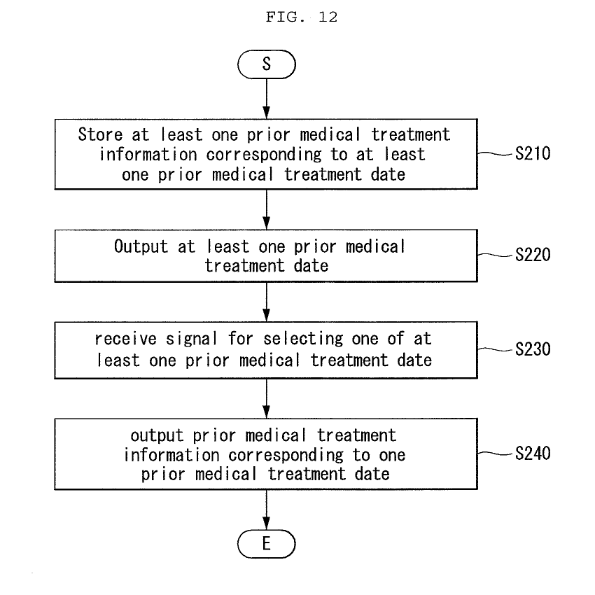

A method for telemedicine according to a first exemplary embodiment will now be described with reference to FIGS. 6 to 11. FIG. 6 is a flow chart illustrating the process of a method for telemedicine according to a first exemplary embodiment of the present invention. FIG. 7 is a first view of outputting an image reflecting a patient in the method for telemedicine according to the first exemplary embodiment of the present invention. FIG. 8 is a second view of outputting an image reflecting a patient in the method for telemedicine according to the first exemplary embodiment of the present invention. FIG. 9 is a view showing an input of a signal for selecting a body part in the method for telemedicine according to the first exemplary embodiment of the present invention. FIG. 10 is a first view showing an input of biometric information in the method for telemedicine according to the first exemplary embodiment of the present invention. FIG. 11 is a second view showing an input of biometric information in the method for telemedicine according to the first exemplary embodiment of the present invention.

As shown in FIG. 6, the method for telemedicine according to a first exemplary embodiment may include at least one of a step (S110) of outputting an image reflecting a patient, a step (S120) of inputting a signal for selecting a body part from the image reflecting the patient, a step (S130) of obtaining biometric information related to a body part, and a step (S140) of outputting the biometric information to a position corresponding to the body part on the image reflecting the patient. Hereinafter, method for telemedicine according to the first exemplary embodiment will now be described.

The apparatus 100 for telemedicine may output an image reflecting a patient (S110).

The output module 120 may output an image reflecting the patient. Here, the image reflecting the patient may include an image showing an actual figure of the patient or a substitute image.

For example, as shown in FIG. 7, the output module 120 may output an image showing an actual figure of the patient. The patient terminal 20 may capture an image of the patient to obtain a video regarding the actual figure of the patient, and transmit the obtained video to the apparatus 100 for telemedicine or the server 10. The communication module 130 may receive the video regarding the actual figure of the patient directly from the patient terminal 20 or through the server 10. The controller 160 may control the output module 120 to output the video which has been received via the communication module 130. In this manner, the output module 120 may output an actual figure of the patient in real time. Thus, the apparatus 100 for telemedicine can provide the actual figure of the patient to the doctor in real time, whereby the doctor at a remote area can treat the patient.

In another example, as shown in FIG. 8, the output module 120 may output a substitute image of the patient. The storage unit 140 may store a substitute image of the patient and the controller 160 may control the output module 120 to output the stored substitute image. In case of displaying an actual figure of the patient, the entire body of the patient may not be displayed, but the substitute image may reflect the entire body of the patient as shown in FIG. 8. Also, the utilization of the substitute image may obtain an effect of protecting the patient's privacy.

The apparatus 100 for telemedicine may receive a signal for selecting a body part from the image reflecting the patient (S120).

The input module 110 may receive a signal for selecting a particular body part of the patient.

For example, the input module 110 may receive a signal for selecting a body part from the image reflecting the patient. As shown in FIGS. 7 and 8, the output module 120 may provide the image reflecting the patient and the input module 110 may receive a signal for selecting a particular position of the displayed image reflecting the patient from the user. In detail, the input module 110 and the output module 120 may be implemented as a touch screen, the touch screen may provide an image reflecting the patient, and the user may touch a particular point on the image reflecting the patient provided on the touch screen. Accordingly, the input module 110 implemented as the touch screen may receive the signal for selecting the body part corresponding to the touched particular point. In a similar manner, the input module 110 may be implemented as a mouse, a keyboard, and the like, and receive a signal for selecting a body part from the image reflecting the patient by manipulating the cursor displayed on the output module 120.

In another example, as shown in FIG. 9, the input module 110 may receive a signal for selecting a body part from a list of body parts. Here, the controller 160 may receive a signal requesting an entry to a body part selection mode via the input module 110 and display the list of body parts including at least one body part via the output module 120. The input module 110 may receive a signal for selecting a particular body part from among the at least one body part included in the body part list.

The apparatus 100 for telemedicine may obtain biometric information related to the body part (S130).

Here, the biometric information may include information regarding a physical condition of the patient obtained by measuring a body part of the patient. For example, the biometric information may include at least one of body temperature, blood sugar level, blood pressure level, pulse, an auscultation sound, a photo image of an affected part, an X-ray photo image, and any other information obvious to the skilled person in the art.

In general, in case of medical treatment, the doctor may recognize the patient's symptom through a physical examination by interview with the patient, and measure the patient's body part with physical examination equipment to make a precise diagnosis to thus obtain biometric information. Also, in case of a patient of a chronic disease such as a metabolic syndrome, high blood pressure, diabetes, and the like, the patient may periodically measure his biometric information such as blood pressure level or a blood sugar level by using a strip or a blood pressure gauge.

The controller 160 may obtain biometric information regarding the patient in various manners.

For example, the storage unit 140 stores biometric information measured from prior medical treatment, and the controller 160 may obtain the biometric information from the storage unit 140. Here, biometric information regarding diverse patients may be stored in the storage unit 140. The controller 160 may select a body part based on a signal received via the input module 110 and obtain biometric information regarding a body part selected from among the biometric information from the storage unit 140.

Here, the biometric information regarding a body part may refer to biometric information measured from the body part. For example, in case of an auscultation sound measured by examining a left chest with a stethoscope, it may be biometric information regarding the left chest. Similarly, a photo image of a set of teeth obtained by capturing an image of the set of teeth may be biometric information regarding the mouth. Or, biometric information regarding a body part may refer to biometric information measured to determine a condition of the body part. For example, biometric information obtained by analyzing urine for a kidney disease checkup may be biometric information regarding a kidney, and diabetes figures obtained by analyzing blood pressure for a diabetes checkup may be biometric information regarding the pancreas.

In another example, the controller 160 may receive biometric information from an external device via the communication module 130. Here, the external device may include at least one of the patient terminal 20, the server 10, and any other devices storing biometric information. For example, physical examination equipment may measure biometric information from the patient, and transmit such information to at least one of the patient terminal 20, a mobile communication terminal, and the server 10. The controller 160 may receive biometric information from such an external device via the communication module 130. In this case, the controller 160 may transmit information reflecting a body part to the external device via the communication module 130, and the external device may transmit biometric information related to the body part accordingly. Or, the controller 160 may collectively receive biometric information stored in an external device from an external device via the communication module 130, determine biometric information related to the body part, and selectively obtain the biometric information.

The apparatus 100 for telemedicine may output biometric information to a position corresponding to a body part on the image reflecting the patient (S140).

The output module 120 may output biometric information in various manners.

For example, as shown in FIG. 10, the output module 120 may display biometric information on a position corresponding to a selected body part of the image reflecting the patient. The controller 160 may control the output module 10 to display the obtained biometric information on a position corresponding to the body part.

In another example, as shown in FIG. 11, the output module 120 may display biometric information on an image reflecting the patient, and display various types of biometric information on a different area.

In displaying biometric information on the output module 120, when a plurality of types of biometric information are provided, the controller 160 may control the output module 120 to display a predetermined number of types of biometric information. For example, the controller 160 may determine a time at which each of various types of biometric information was measured, and display the types of biometric information by a predetermined time in order starting from one which was most recently determined.

The method for telemedicine according to the first exemplary embodiment of the present invention has the effect that because the apparatus 100 for telemedicine provides the biometric information of the patient along with the image of the patient, the doctor can effectively perform telemedicine.

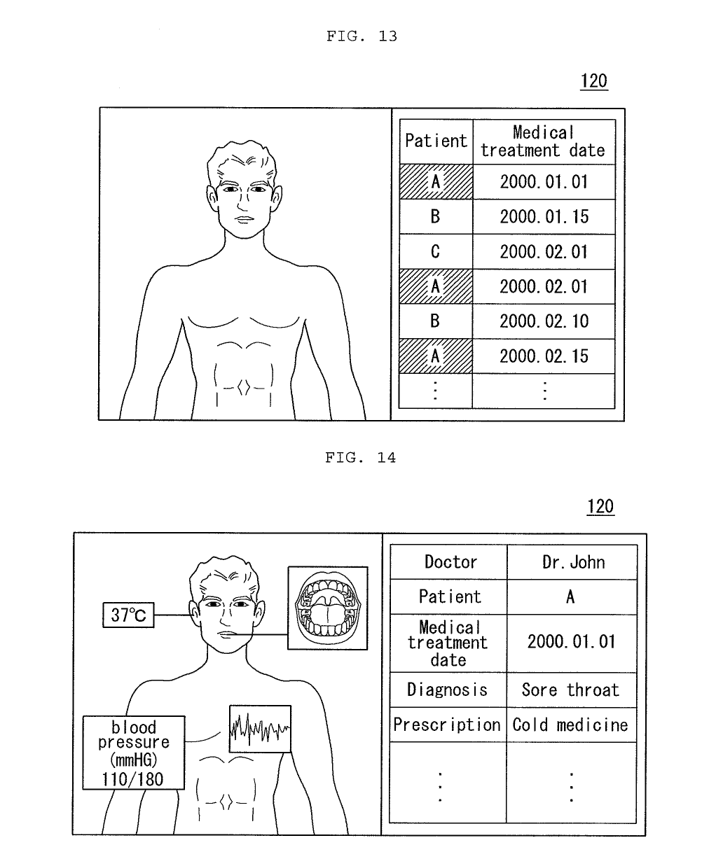

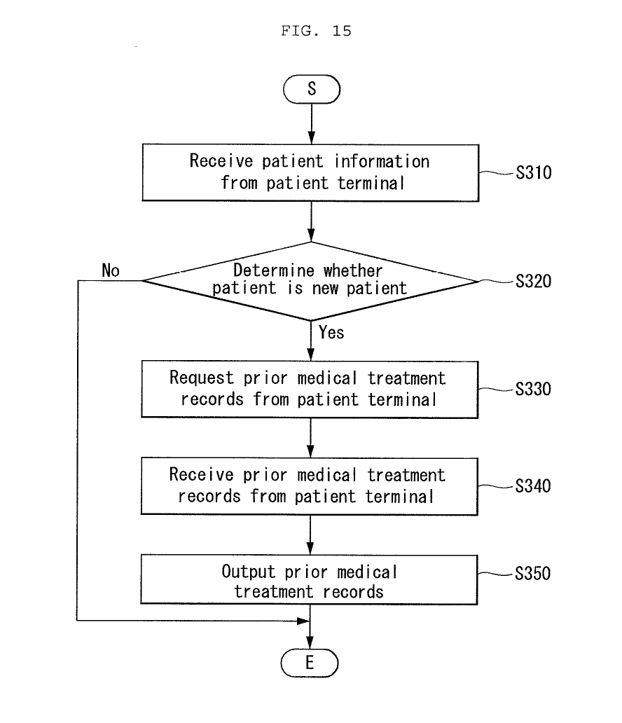

Hereinafter, a method for telemedicine according to a second exemplary embodiment of the present invention will be described with reference to FIGS. 12 to 14. FIG. 12 is a flow chart illustrating the process of a method for telemedicine according to a second exemplary embodiment of the present invention. FIG. 13 is a view showing an output of prior medical treatment history in the method for telemedicine according to the second exemplary embodiment of the present invention. FIG. 14 is a view showing an output of prior medical treatment records in the method for telemedicine according to the second exemplary embodiment of the present invention.

As shown in FIG. 12, the method for telemedicine according to the second exemplary embodiment may include at least one of a step (S210) of storing at least one prior medical treatment information corresponding to at least one prior medical treatment date, a step (S220) of outputting at least one prior medical treatment date, a step (S230) of receiving a signal for selecting one prior medical treatment date among the at least one prior medical treatment date, and a step (S240) of displaying prior medical treatment information corresponding to one medical treatment date.

The apparatus 100 for telemedicine may store at least one prior medical treatment information corresponding to at least one prior medical treatment date (S210).

The storage unit 140 may record medical treatment history. Here, the medical treatment history may refer to history of medical treatment performed in the past. The medical treatment history may include at least one of a person who provides medical treatment, a person who receives medical treatment, a medical treatment date, and medical treatment information. Or, the medical treatment history may not be necessarily stored in the storage unit 140 included in the apparatus 100 for telemedicine. Namely, the medical treatment history may be stored in the server 10 and provided to the apparatus 100 for telemedicine via the communication module 130.

The apparatus 100 for telemedicine may output at least one prior medical treatment date (S220).

The output module 120 may output at least one prior medical treatment date. The controller 160 may control the output module 120 to output medical treatment history stored in the storage unit 140 or medical treatment history obtained through the communication module 130. As shown in FIG. 13, the controller 160 may control the output module 120 to output at least one prior medical treatment date. Here, the controller 160 may control the output module 120 to output to display the patient which received medical treatment at the medical treatment date along with the corresponding prior medical treatment date. Or, the controller 160 may obtain information regarding the patient from an external device, e.g., the patient terminal 20, or the like, via the communication module 130 and control the output module 120 to display only medical treatment history corresponding to the patient according to the information regarding the patient in the medical treatment history. Meanwhile, when video medical treatment is performed by the apparatus 100 for telemedicine, the medical treatment date and the information regarding the patient may be displayed along with an image of the patient. Also, the controller 160 may control the output module 120 to output the prior medical treatment date with respect to the medical treatment corresponding to the patient currently under medical treatment among the medical treatment history.

The apparatus 100 for telemedicine may receive a signal for selecting one prior medical treatment date among at least one prior medical treatment date (S230). The input module 110 may receive a signal for selecting one of the displayed prior medical treatment dates.

The apparatus 100 for telemedicine may display prior medical treatment information corresponding to one prior medical treatment date (S240).

The controller 160 may control the output module 120 to display the prior medical treatment information corresponding to the selected prior medical treatment date based on the signal received via the input module 110. Here, the medical treatment information may include at least one of diagnosis, prescription information, a prescription doctor, a medical treatment time, a determined disease, and biometric information measure through medical examination for a medical treatment.

The controller 160 may display the medical treatment information in various manners via the output module 120. For example, as shown in FIG. 14, the output module 120 may output an image reflecting the patient and bibliographic matters according to the medical treatment information. Also, the output module 120 may display biometric information measured at the corresponding medical treatment date. In this case, the output module 120 may display the biometric information at a position corresponding to a body part related to the biometric information on the image reflecting the patient as shown in FIG. 14.

The method for telemedicine according to the second exemplary embodiment of the present invention has the effect that because the apparatus 100 for telemedicine provides the prior medical treatment history and prior medical treatment information to the doctor, the doctor can review content of the prior medical treatment to effectively perform telemedicine.

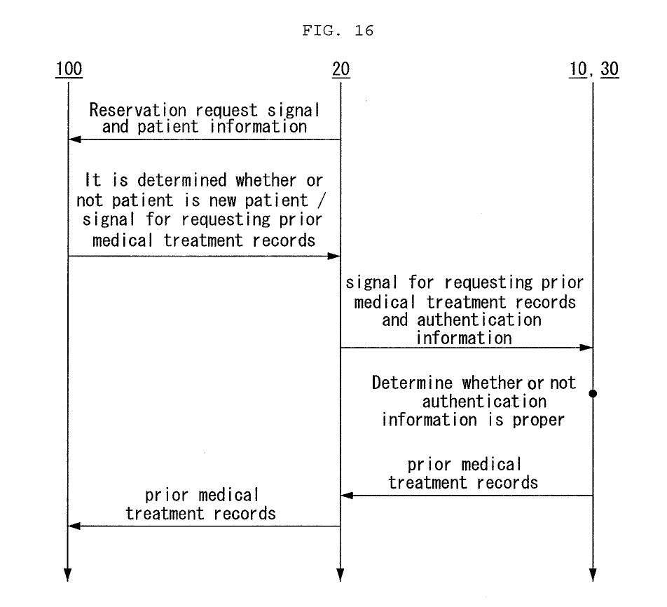

Hereinafter, a method for telemedicine according to a third exemplary embodiment of the present invention will be described with reference to FIGS. 15 and 16. FIG. 15 is a flow chart illustrating the process of a method for telemedicine according to a third exemplary embodiment of the present invention, and FIG. 16 is a signal flow chart of a system for telemedicine in the method for telemedicine according to the third exemplary embodiment of the present invention.

As shown in FIG. 15, the method for telemedicine according to the third exemplary embodiment may comprise at least one of a step (S310) of receiving information regarding a patient from the patient terminal 20, a step (S320) of determining whether or not the patient is a new patient, a step (S330) of requesting prior medical treatment records from the patient terminal 20 when the patient is determined to be a new patient, a step (S340) of receiving prior medical treatment records from the patient terminal 20, and a step (S350) of outputting the prior medical treatment records.

The apparatus 100 for telemedicine may receive information regarding the patient from the patient terminal 20 (S310).

The communication module 130 may receive the information regarding the patient from the patient terminal 20. Here, the information regarding the patient may be information identifying the patient. The communication module 130 may receive the information regarding the patient before or while telemedicine is performed. Alternatively, as shown in FIG. 16, the communication module 130 may receive the information regarding the patient when a signal for requesting a medical treatment reservation is received from the patient terminal 20. Alternatively, when the patient terminal 20 transmits the signal for requesting a medical treatment reservation to the server 10, the communication module 130 may receive the information regarding the patient from the server 10.

The apparatus 100 for telemedicine may determine whether or not the patient is a new patient (or whether or not the patient is subjected to the first medical examination) (S320).

The controller 160 may determine whether or not the patient is a new patient based on the received information regarding the patient. Here, the first medical examination may refer to the fact that the corresponding doctor provides a first medical treatment to the corresponding patient. The case in which the patient received medical treatment from a first doctor and then changed to a second doctor, so the second doctor first provides medical treatment to the patient, may be included in the first medical examination.

Here, the storage unit 140 may store medical treatment history. The medical treatment history may include information regarding a person who has received the medical treatment. The controller 160 may determine whether or not the patient has received medical treatment by comparing the patient according to the information regarding the patient and the person who has received medical treatment according to the medical treatment history. In this manner, the controller 160 may determine whether or not the patient is a new patient.

When the patient is determined to be a new patient, the apparatus 100 for telemedicine may request the prior medical treatment records from the patient terminal 20 (S330).

When the patient is determined to be a new patient according to the determination results, the controller 160 may transmit a signal for requesting the prior medical treatment records to the patient terminal 20.

The patient terminal 20 receives the signal for requesting the prior medical treatment records from the apparatus 100 for telemedicine, and when it retains the prior medical treatment records, the patient terminal 20 may transmit the same to the apparatus 100 for telemedicine.

Alternatively, as shown in FIG. 16, the patient terminal 20 may transmit a signal for requesting the prior medical treatment records from the different apparatus 30 for telemedicine, and the different apparatus 30 for telemedicine may receive the signal and transmit the same to the patient terminal 120 or the apparatus 100 for telemedicine. Upon receiving the prior medical treatment records, the patient terminal 20 may transmit the prior medical treatment records to the apparatus 100 for telemedicine.

Here, the medical treatment records may include every information recorded when the doctor performs medical treatment. For example, the medical treatment records may include a medical treatment date, a medical treatment doctor, a person who receives medical treatment, diagnosis, prescription information, biometric information, and at least one of any other matters recorded by the doctor in the medical treatment. The medical treatment records may be required to have security because the records are personal information. Thus, in order to maintain the security, the medical treatment information may be distributed only to an authenticated person.

In order to transmit and receive the medical treatment records required to have security, as shown in FIG. 16, the patient terminal 20 may transmit the signal for requesting the medical treatment records together with the information regarding the patient and authentication information to the different apparatus 30 for telemedicine. Only when the authentication information is valid, the different apparatus 30 for telemedicine may transmit the medical treatment records to at least one of the patient terminal 20, the apparatus 100 for telemedicine, and the server 10. When the medical treatment records are transmitted to the patient terminal 20, the patient terminal 20 may transmit the medical treatment records to the apparatus 100 for telemedicine.

The apparatus 100 for telemedicine may receive the prior medical treatment records from the patient terminal 20 (S340).

The communication module 130 may receive the prior medical treatment records from the patient terminal 20. Or, the communication module 130 may receive the prior medical treatment records directly from the different apparatus 30 for telemedicine or via the server 10.

The apparatus 100 for telemedicine may output the prior medical treatment records (S350).

The output module 120 may output the prior medical treatment records. The controller 160 may control the output module 120 to output the medical treatment records which have been received via the communication module 130. The controller 160 may display the prior medical treatment records along with an image for a real time medical treatment. Also, when biometric information measured from the patient is included in the prior medical treatment records, the controller 160 may control the output module 120 to display the biometric information on a body part related to the biometric information of the image of the patient.

The method for telemedicine according to the third exemplary embodiment of the present invention has the effect that because the medical treatment records of another doctor are provided to the doctor when a patient to be treated by the doctor is a new patient, the doctor can easily recognize the condition of the patient's health.

The methods for telemedicine according to the respective exemplary embodiments of the present invention can be used alone or may be combined to be used. The steps constituting the respective exemplary embodiments may be used alone or may be combined with the steps constituting the other exemplary embodiments so as to be used.

The exemplary embodiments of the present invention have been described with reference to the accompanying drawings, and it will be apparent to those skilled in the art that various modifications and variations can be made in the present invention without departing from the scope of the invention. Thus, it is intended that any future modifications of the embodiments of the present invention will come within the scope of the appended claims and their equivalents.

What is claimed is:

1. An apparatus for telemedicine, the apparatus comprising:
an output module configured to output a patient image;
an input module configured to receive a signal for selecting a body part; and
a controller configured to:
obtain biometric information related to the body part, and
control the output module to output the biometric information at a position corresponding to the body part on the outputted patient image.

2. The apparatus of claim 1, wherein the signal selecting the body part received by the input unit is generated by selecting a portion of the outputted patient image corresponding to the body part.

3. The apparatus of claim 1, further comprising a communication module configured to communicate with an external device, the external device being configured to obtain the outputted patient image by image capturing,
wherein the controller is configured to receive, via the communication module, the outputted patient image from the external device.

4. The apparatus of claim 1, further comprising a communication module configured to communicate with an external device,
wherein the controller is configured to receive, via the communication module, the biometric information from the external device.

5. The apparatus of claim 1, wherein the controller is configured to control the output module to output a list of at least one body part, and
wherein the signal selecting the body part received by the input unit is generated by selecting the body part from the outputted list of the at least one body part.

6. The apparatus of claim 1, wherein the controller controls the output module to output a predetermined number of the biometric information according to a time of when the measurement of the biometric information occurred.

7. An apparatus for telemedicine, the apparatus comprising:
an input module;
an output module;
a storage unit configured to store at least one prior medical treatment information; and
a controller configured to:
control the output module to display at least one date corresponding to the at least one prior medical treatment information,
receive, via the input module, a signal selecting a date from the at least one displayed date, and
control the output module to display prior medical treatment information corresponding to the selected date.

8. The apparatus of claim 7, further comprising a communication module configured to communicate with an external device,
wherein the controller is configured to receive, via the communication module, patient information, and
wherein the displayed at least one date includes a date when a patient had a medical treatment according to the patient information.

9. The apparatus of claim 7, wherein the prior medical treatment information includes biometric information measured from the patient at the selected date, and
wherein the controller is configured to control the output module to output a patient image and output the biometric information to a position corresponding to a body part related to the biometric information on the patient image.

10. An apparatus for telemedicine, the apparatus comprising:
an output module;
a communication module configured to communicate with an external device;
a storage unit configured to store information; and
a controller configured to:
receive, via the communication module, patient information from a patient terminal, and
determine whether the patient is new based on the existence of any stored prior patient information for the patient, and
if the patient is new, the controller configured to:
transmit, via the communication module, a signal for requesting prior medical treatment records to the patient terminal,
receive, via the communication module, the prior medical treatment records from the patient terminal, and
control the output module to output the received prior medical treatment records.

11. The apparatus of claim 10, wherein the patient terminal transmits a signal for requesting prior medical treatment records to a different apparatus for telemedicine, and receives the prior medical treatment records from the different apparatus for telemedicine.

12. The apparatus of claim 11, wherein the patient terminal transmits authentication information to the different apparatus for telemedicine, and
wherein when the authentication information is correct, the different apparatus for telemedicine transmits the prior medical treatment records to the patient terminal.

13. The apparatus of claim 11, wherein the controller is configured to receive a request for a medical treatment reservation from the patient terminal, and
wherein when the medical treatment reservation is received, the controller is configured to perform the determination of whether the patient is new.

14. A method for providing telemedicine, the method comprising:
outputting, by an output module, a patient image;
receiving a signal selecting a body part;
obtaining biometric information related to the body part; and
outputting the biometric information at a position corresponding to the body part on the outputted patient image.

15. The method of claim 14, further comprising generating the signal by selecting a portion of the outputted patient image corresponding to the body part.

16. The method of claim 14, further comprising:
outputting, by the output module, a list of body parts, and
generating the signal by selecting the body part from the outputted list of body parts.

17. A method for providing telemedicine, the method comprising:
receiving, via a communication module, patient information from a patient terminal,
determining whether the patient is new based on the existence of any stored prior patient information for the patient, and
if the patient is new, transmitting, via the communication module, a signal requesting prior medical treatment records to the patient terminal, receiving, via the communication module, the prior medical treatment records from the patient terminal, and outputting, by an output module, the received prior medical treatment records.
